(19) United States
(12) Patent Application Publication
(10) Pub. No.: US 2010/0210511 A1
(43) Pub. Date: Aug. 19, 2010

(54) Combination VEGFR2 Therapy with Temozolomide

(75) Inventor: Irvith M. Carvajal, Brighton, MA (US)

Correspondence Address: ROPES & GRAY LLP, PATENT DOCKETING Floor 39, One International Place, Boston, MA, 02110-2624, US

(73) Assignee: Bristol-Myers Squibb Company, Princeton, NJ (US)

(21) Appl. No.: 12/703,878

(22) Filed: Feb. 11, 2010

Related U.S. Application Data
(60) Provisional application No. 61/207,376, filed on Feb. 11, 2009.

(51) Int. Cl. A61K 38/16; A61P 35/00

(52) U.S. Cl. 514/8

(57) ABSTRACT

The present disclosure relates to improved methods of treating neoplastic disorders by combining VEGFR2 specific inhibitor treatment with temozolomide. In particular, methods for treating glioblastoma with a combination of a VEGFR2 inhibitor and temozolomide are provided.

### RELATED APPLICATIONS

This application claims the benefit of U.S. Provisional Application Ser. No. 61/207,376, filed Feb. 11, 2009. All of the teachings of the above-referenced provisional application are incorporated herein by reference.

### SEQUENCE LISTING

The instant application contains a Sequence Listing which has been submitted via EFS-Web and is hereby incorporated by reference in its entirety. Said ASCII copy, created on Feb. 11, 2010, is named COTH5271.txt, and is 60,744 bytes in size.

### BACKGROUND OF THE INVENTION

Angiogenesis is the process by which new blood vessels are formed from pre-existing capillaries or post capillary venules; it is an important component of many physiological processes. In cancer, tumor released cytokines or angiogenic factors stimulate vascular endothelial cells by interacting with specific cell surface receptors. The activated endothelial cells secrete enzymes that degrade the basement membrane of the vessels, allowing invasion of the endothelial cells into the tumor tissue. Once situated, the endothelial cells differentiate to form new vessel offshoots of pre-existing vessels. The new blood vessels provide nutrients to the tumor, facilitating further growth, and also provide a route for metastasis. Additional therapies are needed to treat cancer, in particular to inhibit angiogenesis associated with cancer.

### SUMMARY OF THE INVENTION

One aspect of the application provides for a method of treating a subject afflicted with a neoplasm by administering to the subject at least one VEGFR2 specific inhibitor together or in parallel with temozolomide (TMZ) in amounts that together are effective to treat the neoplasm.

The neoplasm may be any abnormal proliferation of cells benign or malignant. In some embodiments, the neoplasm is a solid tumor. In some embodiments, the neoplasm is a cancer. In some embodiments, the neoplasm is dependent on angiogenesis for growth or survival. In some embodiments, the neoplasm is glioblastoma. In certain embodiments, the neoplasm is radiation insensitive, such as, for example, a radiation insensitive glioblastoma.

In some embodiments, the VEGFR2 specific inhibitor and temozolomide are administered sequentially. In some embodiments, the inhibitors are administered together.

In some embodiments, the methods further comprise administration of radiation therapy to the subject. The radiation therapy may be administered together or in parallel with the VEGFR2 specific inhibitor and/or TMZ.

In some embodiments, the VEGFR2 specific inhibitor is selected from an antibody or a fibronectin based scaffold protein.

In some embodiments, methods are provided comprising conjointly administering to a patient in need thereof, temozolomide and a polypeptide comprising a tenth fibronectin type III domain (¹⁰Fn3), wherein the amino acid sequence of the ¹⁰Fn3 is altered in one or more of the BC, DE, or FG loops, relative to the naturally occurring human ¹⁰Fn3 as depicted in SEQ ID NO: 1. In some embodiments, the VEGFR2 binding ¹⁰Fn3 comprises a BC loop having the amino acid sequence set for in residues 14-24 SEQ ID NO: 4, a DE loop having the amino acid sequence set for in residues 44-50 of SEQ ID NO: 4, and an FG loop having the amino acid sequence set for in residues 69-82 of SEQ ID NO: 4. In some embodiments, the VEGFR2 binding ¹⁰Fn3 has an amino acid sequence at least 60, 70, 80, 90, 95, 98, 99, or 100% identical to SEQ ID NO: 4 and comprises a peg moiety of about 40 kDa conjugated to a non-native cysteine residue.

In some embodiments, the VEGFR2 specific inhibitor is a polypeptide comprising an amino acid sequence at least 70, 80, 90, 95, 98, 99, or 100% identical to any one of SEQ ID NOS: 2-62.

### BRIEF DESCRIPTION OF THE DRAWINGS

FIG. 1: Kaplan-Myers Survival Curve of NOD-SCID Mice Intracranially Implanted with U87vIIILuc Glioblastoma Cells. Mice were randomized into vehicle control (Phosphate Buffered Saline (PBS)), TMZ (34 mg/kg QD×5 days), Compound 1 (60 mg/kg×3 times per week) and Combination (Compound 1+TMZ) groups of 6 mice. Y-axis indicates percent survival within each group over the length of the study. Animals were followed for survival and sacrificed at the first sign of morbidity. Compound 1=VEGFR2 specific inhibitor represented by SEQ ID NO: 4; Compound 2=TMZ; PBS=phosphate buffered saline (vehicle control).

FIG. 2: U87vIIILuc Tumor Growth Progression Under Treatment as Measured by Xenogen-Quantitation of Bioluminescence Signal. Bioluminescent imaging (BLI) was performed in a high sensitivity, cooled CCD camera (IVIS®, Xenogen) at five-minute intervals until peak values were recorded for all mice. BLI imaging was performed on days 6, 13, 20 and 26. Convergent arrows indicate maximal BLI as detected for PBS control group. Compound 1=VEGFR2 specific inhibitor represented by SEQ ID NO: 4; Compound 2=TMZ; PBS=phosphate buffered saline (vehicle control).

FIG. 3: U87vIIILuc Tumor Growth Progression Under Treatment as Measured by Xenogen-Quantitation of Mean Bioluminescence Signal. Bioluminescent imaging (BLI) was performed in a high sensitivity, cooled CCD camera (IVIS®, Xenogen) at five-minute intervals until peak values were recorded for all mice. BLI imaging was performed on days 6, 13, 20 and 26. Arrows indicate treatment with Compound 1 while the open square indicates treatment with Compound 2. Compound 1=VEGFR2 specific inhibitor represented by SEQ ID NO: 4; Compound 2=TMZ; PBS=phosphate buffered saline (vehicle control).

FIG. 4: U87vIIILuc Microvascular Density, Proliferation, and Apoptosis Responses Evaluated by Immunohistochemistry Analysis after 14 Days of Treatment. Immunohistochemical staining against CD31 (endothelial blood vessel formation marker), Ki67 (cell proliferation marker) or cleaved-caspase 3 (cellular apoptosis marker) was performed on paraffin-embedded brain tissue sections. Stained tissue sections were examined with low and high-power light microscopy and the entire tumor area was selected for density counts. Combo=Compound 1+TMZ; Compound 1=VEGFR2 specific inhibitor represented by SEQ ID NO: 4; Compound 2=TMZ; PBS=phosphate buffered saline (vehicle control).

FIG. 5: Kaplan-Myers Survival Curve of NOD-SCID Mice Intracranially Implanted with U87vIIILuc Glioblastoma Cells After Treatment with Standard of Care or Standard of Care plus Compound 1. Mice were randomized into vehicle control (Phosphate Buffered Saline (PBS)), Radiation Therapy (RT), Combination Compound 2 & RT, and Combination Compound 1 plus Compound 2 & RT. Y-axis indicates percent survival within each group over the length of the study. Animals were followed for survival and sacrificed at the first sign of morbidity. Compound 1=VEGFR2 specific inhibitor represented by SEQ ID NO: 4; Compound 2=TMZ; PBS=phosphate buffered saline (vehicle control); RT=radiation therapy.

FIG. 6A-D: U87vIIILuc Tumor Growth Progression Under Treatment as Measured by Xenogen-Quantitation of Bioluminescence Signal After Treatment with Standard of Care or Standard of Care plus Compound 1. Bioluminescent imaging (BLI) was performed in a high sensitivity, cooled CCD camera (IVIS®, Xenogen) at five-minute intervals until peak values were recorded for all mice. BLI imaging was performed on days 3, 7, 10, 14, 21, 28, 35, 42, and 49. Convergent arrows indicate maximal BLI as detected for PBS control group. Panel A=combination of RT+Compound 1+Compound 2; Panel B=combination of RT+Compound 2; Panel c=RT alone; Panel D=PBS control. Compound 1=VEGFR2 specific inhibitor represented by SEQ ID NO: 4; Compound 2=TMZ; PBS=phosphate buffered saline (vehicle control); RT=radiation therapy.

FIG. 7: U87vIIILuc Tumor Growth Progression Under Treatment as Measured by Mean Xenogen-Quantitation of Bioluminescence Signal After Treatment with Standard of Care or Standard of Care plus Compound 1. Bioluminescent imaging (BLI) was performed in a high sensitivity, cooled CCD camera (IVIS®, Xenogen) at five-minute intervals until peak values were recorded for all mice. BLI imaging was performed on days 3, 7, 10, and 14. Down-pointing arrows indicate treatment with Compound 1. The closed rectangle indicates treatment with Radiation Therapy. The open rectangle indicates treatment with Compound 2. Compound 1=VEGFR2 specific inhibitor represented by SEQ ID NO: 4; Compound 2=TMZ; PBS=phosphate buffered saline (vehicle control); RT=radiation therapy.

FIG. 8: multiple sequence alignment of exemplary ¹⁰Fn3-based VEGFR2 specific inhibitors (SEQ ID NOs: 2-62) as compared to the wild-type human ¹⁰Fn3 sequence (SEQ ID NO: 1). The SEQ ID NOs are shown in the left column. The BC, DE and FG loops are separated by gaps and are labeled at the top of the figure.

### DETAILED DESCRIPTION OF THE INVENTION

### Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The terms “comprise” and “comprising” are used in the inclusive, open sense, meaning that additional elements may be included.

The term “including” is used to mean “including but not limited to”. “Including” and “including but not limited to” are used interchangeably.

By a “polypeptide” is meant any sequence of two or more amino acids, regardless of length, post-translation modification, or function. “Polypeptide,” “peptide,” and “protein” are used interchangeably herein.

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2. The ALIGN-2 sequence comparison computer program was authored by Genentech, Inc. has been filed with user documentation in the U.S. Copyright Office, Washington D.C., 20559, where it is registered under U.S. Copyright Registration No. TXU510087, and is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

The “half-life” of an amino acid sequence or compound can generally be defined as the time taken for the serum concentration of the polypeptide to be reduced by 50% in vivo due to, e.g., degradation of the sequence or compound and/or clearance or sequestration of the sequence or compound by natural mechanisms. The half-life can be determined in any manner known in the art, such as by pharmacokinetic analysis. See e.g., M Gibaldi & D Perron “Pharmacokinetics”, published by Marcel Dekker, 2nd Rev. edition (1982).

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the time to disease progression (TTP) and/or determining the response rates (RR).

By “treating” is meant to slow the extent or rate of spreading of the cancer, to slow the growth of cancer, to kill or arrest cancer cells that may have spread to other parts of the body from the original tumor, to relieve symptoms caused by the cancer, or to delay the onset of cancer. The symptoms to be relieved using the combination therapies described herein include pain and other types of discomfort.

### Overview

Despite aggressive treatment of malignant glioma, there has been little improvement over the past 30 years in the survival of patients with malignant gliomas. Radiation therapy (RT) remains the mainstay of post-surgical management. Recently, the concurrent use of the oral alkylating agent temozolomide with RT has been shown to modestly increase prognosis in patients who have undergone complete surgical resection (Stupp, R. et al. (2005) N Engl J Med 352:987-996). Temozolomide is a monofunctional alkylating agent with a favorable toxicity profile commonly used in the treatment of malignant glioma. Although the combined use of temozolomide and RT is now a preferred regimen for the treatment of both newly diagnosed and recurrent glioblastoma, the prognosis for people with malignant glioma remains dismal. Promising investigational targeted therapies (Castro, M. G. et al. (2003) Pharmacol Ther 98:71-108), such as targeted toxins, monoclonal antibodies or immune mediated approaches, have yet to make a significant clinical impact. A number of factors account for the poor response of malignant brain tumors to therapy, including the intrinsic resistance of glioma cells to DNA damage-induced cytotoxicity (Taghian, A. et al. (1995) Int J Radiat Oncol Biol Phys 32:99-104) (Johnstone, R. W. et al. (2002) Cell 108:153-164) and the normal tissue toxicity produced by currently employed therapeutic agents.

The disclosure relates, in part, to the surprising discovery that the combination of a VEGFR2 specific inhibitor and temozolomide, or a triple combination of a VEGFR2 specific inhibitor, temozolomide and RT, results in an enhanced survival benefit in an orthotopic model of glioblastoma. The disclosure provides novel methods of treatment and combination therapies to treat neoplasms, in particular angiogenesis dependent neoplasms such as glioblastoma. The novel treatment regimes comprise the administration of at least one VEGFR2 specific inhibitor and temozolomide or a triple combination of at least one VEGFR2 specific inhibitor, temozolomide and RT.

### VEGFR2 Specific Inhibitors

VEGFR2 specific inhibitors useful in the present invention may be any protein or small molecule that specifically binds VEGFR2 and inhibits or reduces one or more VEGFR2 biological functions. By “specifically binds” is meant a molecule that recognizes and interacts with VEGFR2 but that does not substantially recognize and interact with other molecules. In some embodiments, VEGFR2 specific inhibitors bind VEGFR2 with a K_{D} less than 500, 100, 1.0, 0.1, 0.01, or 0.001 nM.

Examples of VEGFR2 specific inhibitors include antibodies, such as heavy chain antibodies, antibodies naturally devoid of light chains, single domain antibodies derived from conventional four-chain antibodies, engineered antibodies and single domain scaffolds other than those derived from antibodies. Examples of VEGFR2 specific inhibitors include CDP-791 (UCB), IMC-1121b (ImClone Systems), and AVE-005 (VEGF trap, Regeneron Pharmaceuticals). Other examples of VEGFR2 specific inhibitors include moieties such as affibodies, afflins, anticalins, avimers, DARPins, microbodies, trans-bodies; or inhibitors that are derived from lipocalins, ankyrins, tetranectins, C-type lectin, Protein A, gamma-crystalline, cysteine knots, and transferrin.

According to one aspect of the invention, a single domain antibody as used herein is a naturally occurring single domain antibody known as heavy chain antibody devoid of light chains. Such single domain antibodies are disclosed in WO 94/04678 for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, dromedary, llama, vicuna, alpaca and guanaco. Other species besides Camelidae may produce heavy chain antibodies naturally devoid of light chain; such VHHs are within the scope of the invention.

VHHs, according to the present invention, and as known to the skilled in the art are heavy chain variable domains derived from immunoglobulins naturally devoid of light chains such as those derived from Camelidae as described in WO 94/04678 (and referred to hereinafter as VHH domains or nanobodies). VHH molecules are about 10 times smaller than IgG molecules. They are single polypeptides and very stable, resisting extreme pH and temperature conditions. Moreover, they are resistant to the action of proteases which is not the case for conventional antibodies. Furthermore, in vitro expression of VHHs produces high yield, properly folded functional VHHs. In addition, antibodies generated in Camelids will recognize epitopes other than those recognized by antibodies generated in vitro through the use of antibody libraries or via immunization of mammals other than Camelids (WO9749805). As such, anti VEGFR2 VHH's may interact more efficiently with VEGFR2 than conventional antibodies, thereby blocking its interaction with the VEGF ligand(s) more efficiently. Since VHH's are known to bind into ‘unusual’ epitopes such as cavities or grooves (WO97/49805), the affinity of such VHH's may be more suitable for therapeutic treatment.

Another example of a VEGFR2 specific inhibitor is anti-VEGFR-2 consisting of a sequence corresponding to that of a Camelidae VHH directed towards VEGFR-2 or a closely related family member. The invention also relates to a homologous sequence, a function portion or a functional portion of a homologous sequence of said polypeptide. The invention also relates to nucleic acids capable of encoding said polypeptides. A single domain antibody of the present invention may be directed against VEGFR-2 or a closely related family member.

The present invention further relates to single domain antibodies of VHH belonging to a class having human-like sequences. One such class is characterized in that the VHHs carry an amino acid from the group consisting of glycine, alanine, valine, leucine, isoleucine, proline, phenylalanine, tyrosine, tryptophan, methionine, serine, threonine, asparagine, or glutamine at position 45, such as, for example, L45 and a tryptophan at position 103, according to the Kabat numbering. As such, polypeptides belonging to this class show a high amino acid sequence homology to human VH framework regions and said polypeptides might be administered to a human directly without expectation of an unwanted immune response therefrom, and without the burden of further humanisation.

Another human-like class of Camelidae single domain antibodies has been described in PCT Publication No. WO03/035694 and contain the hydrophobic FR2 residues typically found in conventional antibodies of human origin or from other species, but compensating this loss in hydrophilicity by the charged arginine residue on position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanization. The invention also relates to nucleic acids capable of encoding said polypeptides. Polypeptides may include the full length Camelidae antibodies, namely Fc and VHH domains.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)₂ fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

Various techniques have been developed for the production of antibody fragments that may be used to make antibody fragments used in the invention. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

### Fn3-Based VEGFR2 Specific Inhibitors

An exemplary VEGFR2 specific inhibitor is based on a fibronectin type III domain (Fn3). Fibronectin is a large protein which plays essential roles in the formation of extracellular matrix and cell-cell interactions; it consists of many repeats of three types (types I, II, and III) of small domains.

Fn3 is small, monomeric, soluble, and stable. It lacks disulfide bonds and, therefore, is stable under reducing conditions. The overall structure of Fn3 resembles the immunoglobulin fold. Fn3 domains comprise, in order from N-terminus to C-terminus, a beta or beta-like strand, A; a loop, AB; a beta or beta-like strand, B; a loop, BC; a beta or beta-like strand, C; a loop, CD; a beta or beta-like strand, D; a loop, DE; a beta or beta-like strand, E; a loop, EF; a beta or beta-like strand, F; a loop, FG; and a beta or beta-like strand, G. The seven antiparallel 13-strands (A through G) are arranged as two beta sheets that form a stable core, while creating two “faces” composed of the loops that connect the beta or beta-like strands. Loops AB, CD, and EF are located at one face and loops BC, DE, and FG are located on the opposing face. Any or all of loops AB, BC, CD, DE, EF and FG may participate in ligand binding. There are at least 15 different modules of Fn3, and while the sequence homology between the molecules is low, they all share a high similarity in tertiary structure. The structure of Fn3 scaffolds and methods for selecting modified scaffolds that bind to a desired target are discussed, for example, in US Patent Publication No. US 2006/0246059 and Binz, et al., Nature Biotechnology 23(10): 1257-1268 (2005).

Adnectins™ (Adnexus, a Bristol-Myers Squibb R&D Company) are ligand binding scaffold proteins based on the tenth fibronectin type III domain, i.e., the tenth module of Fn3, (¹⁰Fn3). The amino acid sequence of a naturally occurring human ¹⁰Fn3 is set forth in SEQ ID NO: 1.

(SEQ ID NO: 1)
VSDVPRDLEVVAATPTSLLISWDAPAVTVRYYRITYGETGGNSPVQEFTV
PGSKSTATISGLKPGVDYTITVYAVTGRGDSPASSKPISINYRT
(BC, FG, and DE loops are emphasized)

In SEQ ID NO: 1, the AB loop corresponds to residues 15-16, the BC loop corresponds to residues 21-30, the CD loop corresponds to residues 39-45, the DE loop corresponds to residues 51-56, the EF loop corresponds to residues 60-66, and the FG loop corresponds to residues 76-87.

¹⁰Fn3 are structurally and functionally analogous to antibodies, specifically the variable region of an antibody. While ¹⁰Fn3 domains may be described as “antibody mimics” or “antibody-like proteins”, they do offer a number of advantages over conventional antibodies. In particular, they exhibit better folding and thermostability properties as compared to antibodies, and they lack disulphide bonds, which are known to impede or prevent proper folding under certain conditions. Exemplary ¹⁰Fn3-based VEGFR2 specific inhibitors are predominantly monomeric with Tm's averaging ˜50° C.

The BC, DE, and FG loops of ¹⁰Fn3 are analogous to the complementary determining regions (CDRs) from immunoglobulins. Alteration of the amino acid sequence in these loop regions changes the binding specificity of ¹⁰Fn3. The protein sequences outside of the CDR-like loops are analogous to the framework regions from immunoglobulins and play a role in the structural conformation of the ¹⁰Fn3. Alterations in the framework-like regions of ¹⁰Fn3 are permissible to the extent that the structural conformation is not so altered as to disrupt ligand binding. Methods for generating ¹⁰Fn3 ligand specific binders have been described in PCT Publication Nos. WO 00/034787, WO 01/64942, and WO 02/032925, disclosing high affinity TNFα binders, PCT Publication No. WO 2008/097497, disclosing high affinity VEGFR2 binders, and PCT Publication No. WO 2008/066752, disclosing high affinity IGFIR binders. Additional references discussing ¹⁰Fn3 binders and methods of selecting binders include PCT Publication Nos. WO 98/056915, WO 02/081497, and WO 2008/031098 and U.S. Publication No. 2003186385.

In some embodiments, a ¹⁰Fn3-based VEGFR2 specific inhibitor has an amino acid sequence at least 40, 50, 60, 70, or 80% identical to the human ¹⁰Fn3 domain, shown in SEQ ID NO: 1. Much of the variability will generally occur in one or more of the loops.

In some embodiments, the disclosure provides ¹⁰Fn3-based VEGFR2 specific inhibitors having at least one loop selected from loop BC, DE, and FG with an altered amino acid sequence relative to the sequence of the corresponding loop of the human ¹⁰Fn3. By “altered” is meant one or more amino acid sequence alterations relative to a template sequence (corresponding human fibronectin domain) and includes amino acid additions, deletions, and substitutions. Altering an amino acid sequence may be accomplished through intentional, blind, or spontaneous sequence variation, generally of a nucleic acid coding sequence, and may occur by any technique, for example, PCR, error-prone PCR, or chemical DNA synthesis. In some embodiments, an amino acid sequence is altered by substituting with or adding naturally occurring amino acids.

In some embodiments, one or more loops selected from BC, DE, and FG may be extended or shortened in length relative to the corresponding human fibronectin loop. In particular, the FG loop of the human ¹⁰Fn3 is 12 residues long, whereas the corresponding loop in antibody heavy chains ranges from 4-28 residues. To optimize antigen binding, therefore, the length of the FG loop of ¹⁰Fn3 may be altered in length as well as in sequence to obtain the greatest possible flexibility and affinity in antigen binding.

In some embodiments of the ¹⁰Fn3 molecules, the altered BC loop has up to 10 amino acid substitutions, up to 9 amino acid deletions, up to 10 amino acid insertions, or a combination of substitutions and deletions or insertions. In some embodiments, the altered DE loop has up to 6 amino acid substitutions, up to 5 amino acid deletions, up to 14 amino acid insertions or a combination of substitutions and deletions or insertions. In some embodiments, the FG loop has up to 12 amino acid substitutions, up to 11 amino acid deletions, up to 28 amino acid insertions or a combination of substitutions and deletions or insertions.

Naturally occurring ¹⁰Fn3 comprises an “arginine-glycine-aspartic acid” (RGD) integrin-binding motif in the FG loop. Preferred VEGFR2 specific binders lack an RGD integrin-binding motif. The RGD binding motif may be removed or disrupted by any suitable method. For example, one or more of the R, G or D residues may be deleted. Alternatively, one or more amino acids may be inserted between the R and the G and/or between the G and the D residues. In yet another embodiment, one or more of the R, G or D residues may be substituted for another amino acid. In an exemplary embodiment, all three of the R, G and D residues are substituted with other amino acid residues.

¹⁰Fn3 generally begin with the amino acid residue corresponding to number 1 of SEQ ID NO: 1. However, domains with amino acid deletions are also encompassed by the invention. In some embodiments, amino acid residues corresponding to the first eight amino acids of SEQ ID NO: 1 are deleted. Additional sequences may also be added to the N- or C-terminus. For example, an additional MG sequence may be placed at the N-terminus of ¹⁰Fn3. The M will usually be cleaved off, leaving a G at the N-terminus. In some embodiments, sequences may be placed at the C-terminus of the ¹⁰Fn3 domain, e.g., EIDKPSQ (SEQ ID NO: 68) or EIDKPCQ (SEQ ID NO: 69)

The non-ligand binding sequences of ¹⁰Fn3, i.e., the “¹⁰Fn3 scaffold”, may be altered provided that the ¹⁰Fn3 retains ligand binding function and structural stability. In some embodiments, one or more of Asp 7, Glu 9, and Asp 23 are replaced by another amino acid, such as, for example, a non-negatively charged amino acid residue (e.g., Asn, Lys, etc.). These mutations have been reported to have the effect of promoting greater stability of the mutant ¹⁰Fn3 at neutral pH as compared to the wild-type form (See, PCT Publication No. WO 02/04523). A variety of additional alterations in the ¹⁰Fn3 scaffold that are either beneficial or neutral have been disclosed. See, for example, Batori et al., Protein Eng. 2002 15(12):1015-20; Koide et al., Biochemistry 2001 40(34):10326-33.

The ¹⁰Fn3 scaffold may be modified by one or more conservative substitutions. As many as 5%, 10%, 20% or even 30% or more of the amino acids in the ¹⁰Fn3 scaffold may be altered by a conservative substitution without substantially altering the affinity of the ¹⁰Fn3 for a ligand. It may be that such changes will alter the immunogenicity of the ¹⁰Fn3 in vivo, and where the immunogenicity is decreased, such changes will be desirable. As used herein, “conservative substitutions” are residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., Atlas of Protein Sequence and Structure 5:345-352 (1978 & Supp.). Examples of conservative substitutions are substitutions within the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine.

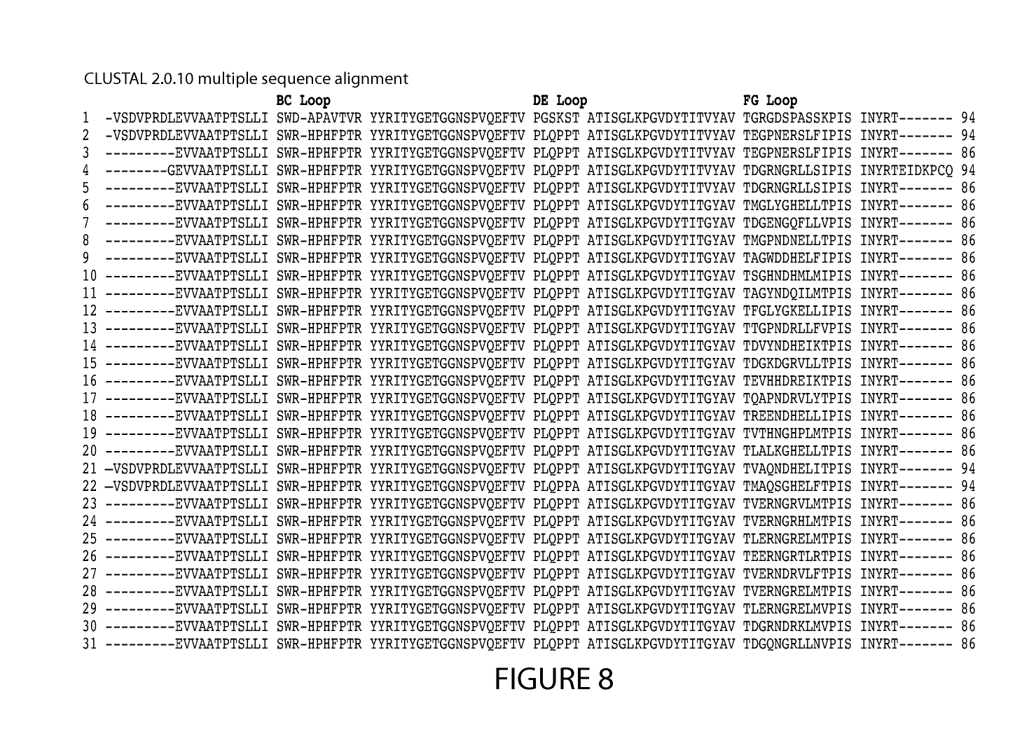

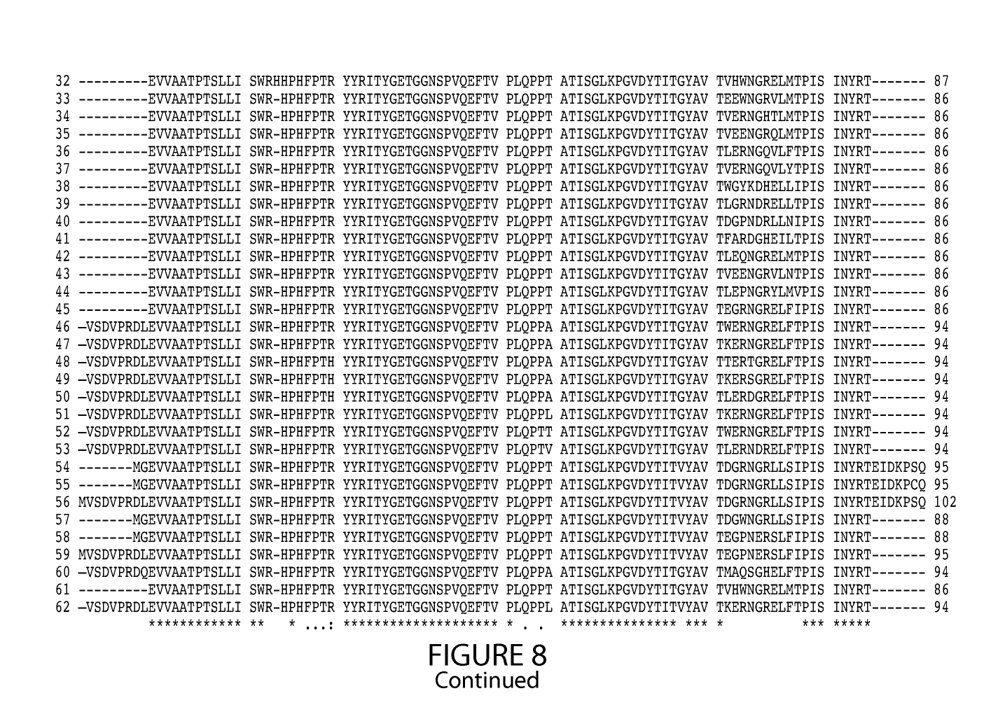

Working examples of ¹⁰Fn3-based VEGFR2 specific inhibitors were generated as described in PCT Publication No. WO 2005/056764, which is hereby incorporated by reference. Sequences of exemplary polypeptides useful for the invention are SEQ ID NOs: 2-62 shown in FIG. 8. In some embodiments, the VEGFR-2 specific inhibitor is a polypeptide comprising an amino acid sequence at least 60, 70, 80, 85, 90, 95, 98, or 100% identical to any one of SEQ ID NOs: 2-62.

In some embodiments, the VEGFR-2 specific inhibitor is a polypeptide comprising an amino acid sequence having SEQ ID NO: 63: EVVAATPTSLLISWRHPHFPTX₁YYRITYGETGGNSPVQEFTVPLQPX₂X₃ATISGLKPGVD YTITGYAX₄TX₅X₆X₇X₈X₉X₁₀X₁₁X₁₂X₁₃X₁₄PISINYRT (SEQ ID NO: 63). In certain embodiments, wherein X₁ is R or H, X₂ is P or T, X₃ is A, L, T or V, X₄ is G or V, and X₅ through X₁₄ are any amino acid. In certain embodiments, wherein X₁ is R or H, X₂ is P or T, X₃ is A, L, T or V, X₄ is G or V, X₅ is L or M, X₆ is G, X₇ is any amino acid, X₈ is N, X₉ is G or D, X₁₀ is H or R, X₁₁ is E, X₁₂ is L, X₁₃ is L or M, and X₁₄ is T. In certain embodiments, wherein X₁ is R or H, X₂ is P or T, X₃ is A, L, T or V, X₄ is G or V, X₅ is any amino acid, X₆ is E, X₇ is R, X₈ is N, X₉ is G, X₁₀ is R, X₁₁ is any amino acid, X₁₂ is L, X₁₃ is Lm M or N, and X₁₄ is T. In certain embodiments, wherein X₁ is R or H, X₂ is P or T, X₃ is A, L, T or V, X₄ is G or V, X₅ is D or E, X₆ is G, X₇ is any amino acid, X₈ is N, X₉ is any amino acid, X₁₀ is R, X₁₁ is any amino acid, X₁₂ is L, X₁₃ is any amino acid, and X₁₄ is I. In certain embodiments, wherein X₁ is R or H, X₂ is P or T, X₃ is A, L, T or V, X₄ is G or V, X₅ is D or E, X₆ is G, X₇ is R or P, X₈ is N, X₉ is G or E, X₁₀ is R, X₁₁ is S or L, X₁₂ is L, X₁₃ is S or F, and X₁₄ is I.

In certain embodiments, the VEGR-2 specific inhibitor comprises an FG loop having a sequence set forth in SEQ ID NOs: 64-67: (L/M)GXN(G/D)(H/R)EL(L/M)TP (SEQ ID NO: 64), XERNGRXL(L/M/N)TP (SEQ ID NO: 65), (D/E)GXNXRXLXIP (SEQ ID NO: 66), (D/E)G(R/P)N(G/E)R(S/L)L(S/F)IP (SEQ ID NO: 67), wherein X can be any amino acid and (/) represents alternative amino acid for the same position.

In some embodiments, the VEGFR-2 specific inhibitor is a ¹⁰Fn3 based protein comprising a BC loop having the amino acid sequence set forth in amino acids 14-24 of SEQ ID NO: 4, a DE loop having the amino acid sequence set forth in amino acids 44-50 of SEQ ID NO: 4, and an FG loop having the amino acid sequence set forth in amino acids 69-82 of SEQ ID NO: 4.

### Pharmacokinetic Moieties

VEGFR2 specific inhibitors useful for the methods of the invention may further comprise a pharmacokinetic (PK) moiety. Improved pharmacokinetics may be assessed according to the perceived therapeutic need. Often it is desirable to increase bioavailability and/or increase the time between doses, possibly by increasing the time that a protein remains available in the serum after dosing. In some instances, it is desirable to improve the continuity of the serum concentration of the protein over time (e.g., decrease the difference in serum concentration of the protein shortly after administration and shortly before the next administration). VEGFR2 specific inhibitors may be attached to a moiety that reduces the clearance rate of the polypeptide in a mammal (e.g., mouse, rat, or human) by greater than three-fold relative to the unmodified polypeptide. Other measures of improved pharmacokinetics may include serum half-life, which is often divided into an alpha phase and a beta phase. Either or both phases may be improved significantly by addition of an appropriate moiety.

Moieties that tend to slow clearance of a protein from the blood include polyoxyalkylene moieties (e.g., polyethylene glycol); sugars (e.g., sialic acid); and well-tolerated protein moieties (e.g., Fc, Fc fragments, transferrin, or serum albumin).

In some embodiments, the PK moiety is a serum albumin binding protein such as those described in U.S. Publication Nos. 2007/0178082 and 2007/0269422.

In some embodiments, the PK moiety is a serum immunoglobulin binding protein such as those described in U.S. Publication No. 2007/0178082.

In some embodiments, the PK moiety is polyethylene glycol (PEG).

The serum clearance rate of a PK-modified VEGFR2 specific inhibitor may be decreased by about 10%, 20%, 30%, 40%, 50%, 60%, 70%, 80%, or even 90%, relative to the clearance rate of the unmodified inhibitor. The PK-modified inhibitor may have a half-life (t_{1/2}) which is enhanced relative to the half-life of the unmodified inhibitor. The half-life of PK-modified inhibitor may be enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the unmodified inhibitor. In some embodiments, the inhibitor half-life is determined in vitro, such as in a buffered saline solution or in serum. In other embodiments, the inhibitor half-life is an in vivo half life, such as the half-life of the inhibitor in the serum or other bodily fluid of an animal.

In some embodiments, a ¹⁰Fn3-based VEGFR2 specific inhibitor binds to VEGFR2 with a K_{D} of less than 100 nM and has a clearance rate of less than 30 mL/hr/kg in a mammal. In some embodiments, the ¹⁰Fn3-based VEGFR2 specific inhibitor comprises a non-native cysteine residue conjugated to a PEG moiety.

In some embodiments, a PK moiety is linked to a VEGFR2 specific inhibitor via at least one disulfide bond, a peptide bond, a polypeptide, a polymeric sugar, or a polyethylene glycol moiety. In some embodiments, the VEGFR2 specific inhibitor is a ¹⁰Fn3-based VEGFR2 specific inhibitor, the PK moiety is a PEG moiety, and exemplary polypeptide linkers include PSTSTST (SEQ ID NO: 70), EIDKPSQ (SEQ ID NO: 68), and GS linkers, such as GSGSGSGSGS (SEQ ID NO: 71) and multimers thereof.

### Polymer Conjugation

Conjugation to a biocompatible polymer may be used to improve the pharmacokinetics or decrease immunogenicity of VEGFR2 specific inhibitors. The identity, size and structure of the polymer is selected so as to improve the circulation half-life of the inhibitor or decrease the antigenicity of the inhibitor without an unacceptable decrease in activity.

Examples of polymers include, but are not limited to, poly(alkylene glycols) such as polyethylene glycol (PEG). The polymer is not limited to a particular structure and can be linear (e.g., alkoxy PEG or bifunctional PEG), or non-linear such as branched, forked, multi-armed (e.g., PEGs attached to a polyol core), and dendritic.

Typically, PEG and other water-soluble polymers (i.e., polymeric reagents) are activated with a suitable activating group appropriate for coupling to a desired site on the polypeptide. Thus, a polymeric reagent will possess a reactive group for reaction with the polypeptide. Representative polymeric reagents and methods for conjugating these polymers to an active moiety are well-known in the art and further described in Zalipsky, S., et al., “Use of Functionalized Poly(Ethylene Glycols) for Modification of Polypeptides” in Polyethylene Glycol Chemistry: Biotechnical and Biomedical Applications, J. M. Harris, Plenus Press, New York (1992), and in Zalipsky (1995) Advanced Drug Reviews 16: 157-182.

Typically, the weight-average molecular weight of the polymer is from about 100 Daltons to about 150,000 Daltons. Exemplary weight-average molecular weights for the biocompatible polymer include about 20,000 Daltons, about 40,000 Daltons, about 60,000 and about 80,000 Daltons. Branched versions of the biocompatible polymer having a total molecular weight of any of the foregoing can also be used.

In some embodiments, the polymer is PEG. PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X—O(CH₂CH₂O)ₙ₋₁CH₂CH₂OH, where n is 20 to 2300 and X is H or a terminal modification, e.g., a C₁₋₄ alkyl. PEG can contain further chemical groups which are necessary for binding reactions, which result from the chemical synthesis of the molecule; or which act as a spacer for optimal distance of parts of the molecule. In addition, such a PEG can consist of one or more PEG side-chains which are linked together. PEGs with more than one PEG chain are called multiarmed or branched PEGs. Branched PEG are described in, for example, European Published Application No. 473084A and U.S. Pat. No. 5,932,462.

To effect covalent attachment of the polymer molecule(s) to a polypeptide, the hydroxyl end groups of the polymer molecule must be provided in activated form, i.e. with reactive functional groups. Suitably activated polymer molecules are commercially available, e.g. from Nektar Therapeutics, Inc., Huntsville, Ala., USA; PolyMASC Pharmaceuticals plc, UK; or SunBio Corporation, Anyang City, South Korea. Alternatively, the polymer molecules can be activated by conventional methods known in the art, e.g. as disclosed in WO 90/13540. Specific examples of activated PEG polymers include the following linear PEGs: NHS-PEG, SPA-PEG, SSPA-PEG, SBA-PEG, SS-PEG, SSA-PEG, SC-PEG, SG-PEG, SCM-PEG, NOR-PEG, BTC-PEG, EPDX-PEG, NCO-PEG, NPC-PEG, CDI-PEG, ALD-PEG, TRES-PEG, VS-PEG, OPSS-PEG, IODO-PEG, and MAL-PEG, and branched PEGs, such as PEG2-NHS, PEG2-MAL, and those disclosed in U.S. Pat. No. 5,932,462 and U.S. Pat. No. 5,643,575, both of which are incorporated herein by reference.

In some embodiments where PEG molecules are conjugated to cysteine residues, the cysteine residues are native to the protein, whereas in other embodiments, one or more cysteine residues are engineered into the protein. Mutations may be introduced into a protein coding sequence to generate cysteine residues. This might be achieved, for example, by mutating one or more amino acid residues to cysteine. Preferred amino acids for mutating to a cysteine residue include serine, threonine, alanine and other hydrophilic residues. Preferably, the residue to be mutated to cysteine is a surface-exposed residue. Algorithms are well-known in the art for predicting surface accessibility of residues based on primary sequence or a protein. Alternatively, surface residues may be predicted by comparing the amino acid sequences of binding polypeptides, given that the crystal structure of the framework based on which binding polypeptides are designed and evolved has been solved (see Himanen et al., Nature. (2001) 20-27; 414(6866):933-8) and thus the surface-exposed residues identified. In some embodiments, cysteine residues are introduced into ¹⁰Fn3-based VEGFR2 specific inhibitors at or near the N- and/or C-terminus, or within loop regions. Pegylation of cysteine residues may be carried out using, for example, PEG-maleiminde, PEG-vinylsulfone, PEG-iodoacetamide, or PEG-orthopyridyl disulfide.

Conventional separation and purification techniques known in the art can be used to purify PEGylated proteins, such as size exclusion (e.g., gel filtration) and ion exchange chromatography. Products may also be separated using SDS-PAGE. Products that may be separated include mono-, di-, tri- poly- and un-pegylated binding polypeptide, as well as free PEG. The percentage of mono-PEG conjugates can be controlled by pooling broader fractions around the elution peak to increase the percentage of mono-PEG in the composition. About ninety percent mono-PEG conjugates represents a good balance of yield and activity.

### Vectors & Polynucleotides Embodiments

Also included in the present disclosure are nucleic acid sequences encoding any of the proteins described herein. As appreciated by those skilled in the art, because of third base degeneracy, almost every amino acid can be represented by more than one triplet codon in a coding nucleotide sequence. In addition, minor base pair changes may result in a conservative substitution in the amino acid sequence encoded but are not expected to substantially alter the biological activity of the gene product. Therefore, a nucleic acid sequence encoding a protein described herein may be modified slightly in sequence and yet still encode its respective gene product.

Nucleic acids encoding any of the various VEGFR2 specific inhibitors disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc Natl Acad Sci USA. 2003 100(2):438-42; Sinclair et al. Protein Expr Purif. 2002 (1):96-105; Connell N D. Curr Opin Biotechnol. 2001 (5):446-9; Makrides et al. Microbiol. Rev. 1996 60(3):512-38; and Sharp et al. Yeast. 1991 7(7):657-78.

General techniques for nucleic acid manipulation are within the purview of one skilled in the art and are also described for example in Sambrook et al., Molecular Cloning: A Laboratory Manual, Vols. 1-3, Cold Spring Harbor Laboratory Press, 2 ed., 1989, or F. Ausubel et al., Current Protocols in Molecular Biology (Green Publishing and Wiley-Interscience: New York, 1987) and periodic updates, herein incorporated by reference. The DNA encoding a protein is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The ability to replicate in a host, usually conferred by an origin of replication, and a selection gene to facilitate recognition of transformants are additionally incorporated. Suitable regulatory elements are well-known in the art.

The proteins described herein may be produced as a fusion protein with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. For prokaryotic host cells that do not recognize and process a native signal sequence, the signal sequence is substituted by a prokaryotic signal sequence selected, for example, from the group of the alkaline phosphatase, penicillinase, 1 pp, or heat-stable enterotoxin II leaders. For yeast secretion, the native signal sequence may be substituted by, e.g., the yeast invertase leader, a factor leader (including Saccharomyces and Kluyveromyces alpha-factor leaders), or acid phosphatase leader, the C. albicans glucoamylase leader, or the signal described in PCT Publication No. WO 90/13646. In mammalian cell expression, mammalian signal sequences as well as viral secretory leaders, for example, the herpes simplex gD signal, are available. The DNA for such precursor regions may be ligated in reading frame to DNA encoding the protein.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

Expression and cloning vectors usually contain a promoter that is recognized by the host organism and is operably linked to the nucleic acid encoding the protein of the invention, e.g., a fibronectin-based scaffold protein. Promoters suitable for use with prokaryotic hosts include the phoA promoter, beta-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the protein of the invention.

Expression vectors used in eukaryotic host cells (e.g., yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the multivalent antibody. One useful transcription termination component is the bovine growth hormone polyadenylation region. See PCT Publication No. WO 94/11026 and the expression vector disclosed therein.

The recombinant DNA can also include sequence encoding for a protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, New York, 1985), the relevant disclosure of which is hereby incorporated by reference.

The expression construct is introduced into the host cell using a method appropriate to the host cell, as will be apparent to one of skill in the art. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells include prokaryotes, yeast, mammalian cells, or bacterial cells. Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988). In some instance it will be desired to produce proteins in vertebrate cells, such as for glycosylation, and the propagation of vertebrate cells in culture (tissue culture) has become a routine procedure. Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. For many applications, the small size of the ¹⁰Fn3-based VEGFR2 specific inhibitors described herein would make E. coli the preferred method for expression.

### Protein Production

Host cells are transformed with the herein-described expression or cloning vectors for protein production and cultured in conventional nutrient media modified as appropriate for inducing promoters, selecting transformants, or amplifying the genes encoding the desired sequences. Suitable host cells for production of the VEGFR-2 specific inhibitors described herein are include prokaryotic, yeast, or higher eukaryotic cells.

Suitable prokaryotes for protein production include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

Eukaryotic host cells used to produce the proteins of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Proteins disclosed herein can also be produced using cell-translation systems. For such purposes, the nucleic acids encoding the proteins must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system).

Proteins disclosed herein can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

The proteins disclosed herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, proteins may be exchanged into different buffers and/or concentrated by any of a variety of methods known to the art, including, but not limited to, filtration and dialysis.

The purified proteins are preferably at least 85% pure, more preferably at least 95% pure, and most preferably at least 98% pure. Regardless of the exact numerical value of the purity, the proteins are sufficiently pure for use as a pharmaceutical product.

### Therapeutic Uses

The present invention provides methods of treating a neoplasm in a subject in need thereof including administering to the patient at least one VEGFR2 specific inhibitor, in particular a ¹⁰Fn3-based inhibitor, together or in parallel with temozolomide in amounts that together are effective to treat said neoplasm. The present invention also provides methods of treating a neoplasm in a subject in need thereof including administering to the patient at least one VEGFR2 specific inhibitor, in particular a ¹⁰Fn3-based inhibitor, together or in parallel with temozolomide and radiation therapy in amounts that together are effective to treat said neoplasm. Neoplasia disorders include, but are not limited to, acral lentiginous melanoma, actinic keratoses, adenocarcinoma, adenoid cycstic carcinoma, adenomas, adenosarcoma, adenosquamous carcinoma, adrenocortical carcinoma, AIDS-related lymphoma, anal cancer, astrocytic tumors, bartholin gland carcinoma, basal cell carcinoma, biliary tract cancer, bone cancer, bile duct cancer, bladder cancer, brain stem glioma, brain tumors, breast cancer, bronchial gland carcinomas, capillary carcinoma, carcinoids, carcinoma, carcinosarcoma, cavernous, central nervous system lymphoma, cerebral astrocytoma, cervical cancer, connective tissue cancer, cholangiocarcinoma, chondosarcoma, choriod plexus papilloma/carcinoma, clear cell carcinoma, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal, epitheloid, esophageal cancer, Ewing's sarcoma, extragonadal germ cell tumor, eye cancer, fibrolamellar, focal nodular hyperplasia, gallbladder cancer, gastric cancer, gastrinoma, germ cell tumors, gestational trophoblastic tumor, glioblastoma, glioma, glucagonoma, head and neck cancer, hemangiblastomas, hemangioendothelioma, hemangiomas, hepatic adenoma, hepatic adenomatosis, hepatocellular carcinoma, leukemias including but not limited to acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to chronic myelocytic (granulocytic) leukemia, chronic lymphocytic leukemia, hairy cell leukemia, lymphomas such as non-Hodgkin's lymphoma and Hodgkin's lymphoma, hypopharyngeal cancer, hypothalamic and visual pathway glioma, childhood, insulinoma, intaepithelial neoplasia, interepithelial squamous cell neoplasia, intraocular melanoma, intra-epithelial neoplasm, invasive squamous cell carcinoma, large cell carcinoma, islet cell carcinoma, Kaposi's sarcoma, kidney cancer, laryngeal cancer, leiomyosarcoma, lentigo maligna melanomas, leukemia-related disorders, lip and oral cavity cancer, liver cancer, lung cancer, lymphoma, malignant mesothelial tumors, malignant thymoma, medulloblastoma, medulloepithelioma, melanoma, meningeal, merkel cell carcinoma, mesothelial, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, neuroepithelial adenocarcinoma nodular melanoma, non-small cell lung cancer, oat cell carcinoma, oligodendroglial, oral cancer, oropharyngeal cancer, osteosarcoma, pancreatic polypeptide, ovarian cancer, ovarian germ cell tumor, pancreatic cancer, papillary serous adenocarcinoma, pineal cell, pituitary tumors, plasmacytoma, pseudosarcoma, pulmonary blastoma, parathyroid cancer, penile cancer, pheochromocytoma, pineal and supratentorial primitive neuroectodermal tumors, pituitary tumor, plasma cell neoplasm, pleuropulmonary blastoma, prostate cancer, rectal cancer, renal cell carcinoma, cancer of the respiratory system, retinoblastoma, rhabdomyosarcoma, sarcoma, serous carcinoma, skin cancer, small cell carcinoma, small intestine cancer, soft tissue carcinomas, somatostatin-secreting tumor, squamous carcinoma, squamous cell carcinoma, stomach cancer, stromal tumors, submesothelial, superficial spreading melanoma, supratentorial primitive neuroectodermal tumors, testicular cancer, thyroid cancer, undifferentiatied carcinoma, urethral cancer, uterine sarcoma, uveal melanoma, verrucous carcinoma, vaginal cancer, vipoma, vulvar cancer, Waldenstrom's macroglobulinemia, well differentiated carcinoma, and Wilm's tumor. In some embodiments, the combination of a VEGFR2 specific inhibitor and temozolomide is used to treat glioblastoma. In some embodiments, the combination of a VEGFR2 specific inhibitor, temozolomide and radiation therapy is used to treat glioblastoma.

In certain embodiments, the combination therapies of the invention may be used to treat a radiation insensitive neoplasm, such as a radiation insensitive glioblastoma.

### Formulation and Administration

Therapeutic formulations useful in the disclosed methods are prepared for storage by mixing the described inhibitors having the desired degree of purity with optional physiologically acceptable carriers, excipients or stabilizers (Remington's Pharmaceutical Sciences 16th edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The active ingredients may also be entrapped in microcapsules prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes. The formulations are preferably pyrogen free.

Sustained-release preparations may be prepared. Suitable examples of sustained-release preparations include semipermeable matrices of solid hydrophobic polymers containing the proteins of the invention, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated proteins of the invention may remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

While the skilled artisan will understand that the dosage of each therapeutic agent will be dependent on the identity of the agent, the preferred dosages can range from about 10 mg/square meter to about 2000 mg/square meter, more preferably from about 50 mg/square meter to about 1000 mg/square meter.

The therapeutic compounds, e.g., a VEGFR2 specific inhibitor and temozolomide, are administered to a subject, in a pharmaceutically acceptable dosage form. They can be administered intravenously as a bolus or by continuous infusion over a period of time, by intramuscular, subcutaneous, intra-articular, intrasynovial, intrathecal, oral, topical, or inhalation routes. The compounds may also be administered by intratumoral, peritumoral, intralesional, or perilesional routes, to exert local as well as systemic therapeutic effects. Suitable pharmaceutically acceptable carriers, diluents, and excipients are well known and can be determined by those of skill in the art as the clinical situation warrants. Examples of suitable carriers, diluents and/or excipients include: (1) Dulbecco's phosphate buffered saline, pH about 7.4, containing about 1 mg/ml to 25 mg/ml human serum albumin, (2) 0.9% saline (0.9% w/v NaCl), and (3) 5% (w/v) dextrose. The method of the present invention can be practiced in vitro, in vivo, or ex vivo. The compounds can be in the formulation in a concentration of from 1 to 15 mg/ml. In one embodiment, the formulations are administered intravenously. Suitable pharmaceutically acceptable carriers, diluents, and excipients for co-administration will be understood by the skilled artisan to depend on the identity of the particular therapeutic agent being co-administered.

When present in an aqueous dosage form, rather than being lyophilized, the compounds typically will be formulated, together or independently, at a concentration of about 0.1 mg/ml to 100 mg/ml, although wide variation outside of these ranges is permitted. For the treatment of disease, the appropriate dosage of the compounds will depend on the type of disease to be treated, as defined above, the severity and course of the disease, whether the compounds are administered for preventive or therapeutic purposes, the course of previous therapy, the patient's clinical history and response to the compounds, and the discretion of the attending physician. The compounds are suitably administered to the patient at one time or over a series of treatments.

Depending on the type and severity of the disease, preferably from about 1 mg/square meter to about 2000 mg/square meter of compounds is an initial candidate dosage for administration to the patient, more preferably from about 10 mg/square meter to about 1000 mg/square meter of inhibitor whether, for example, by one or more separate administrations, or by continuous infusion. For repeated administrations over several days or longer, depending on the condition, the treatment is repeated until a desired suppression of disease symptoms occurs. However, other dosage regimens may be useful and are not excluded.

In some embodiments, the compounds are subcutaneously administered. The compounds are formulated, together or separately, into pharmaceutically acceptable compositions and may be administered twice daily, once daily, on alternative days, or weekly. In some embodiments, the compounds are administered between 0.5 mg/kg to 2 mg/kg. In some embodiments, the compounds are administered at 0.1, 0.2, 0.3, or 0.4 mg/kg daily. In some embodiments, the patient is first administered an IV load of compounds, for example from 0.5 to 2 mg/kg.

Temozolomide is commercially available, e.g., as Temodar™ capsules (Schering-Plough Corporation). Processes for preparing temozolomide are also described in US Publication Nos. 20070225496 and 20050187206. While temozolomide is commonly administered orally in capsule form, it should be appreciated that temozolomide could be also be administered by any other suitable means, e.g., intraperitoneally. PCT Publication No. WO03/072082 discloses pharmaceutical formulations comprising temozolomide for intravenous administration. US Publication No. 20060122162 discloses pharmaceutical formulations comprising temozolomide for intrathecal administration.

The VEGFR2 specific inhibitor and temozolomide are administered to a patient conjointly. The compounds may be administered in parallel, i.e., they are administered as separate pharmaceutical compositions. They may be administered at the same time or sequentially. The dosage schedule of the compounds may be different, although overlapping in time. Alternatively, the compounds may be administered together, i.e., in a single pharmaceutical composition. In some embodiments, temozolomide and the VEGFR2 specific inhibitors are administered in parallel within five days of each other, 24 hours, 12 hours, or 6 hours of each other. In an exemplary embodiment, Temozolomide is administered orally and the VEGFR2 specific inhibitor is administered intraperitoneally or intravenously.

The methods of the invention may involve administering radiation therapy to a subject in addition to the VEGFR2 specific inhibitor and Temozolomide. Radiation therapy includes ionizing radiation (e.g., x-radiation, gamma-radiation, visible radiation, ultraviolet light, radiation, infrared radiation, microwave radiation) and radioactive isotope therapies. Examples of radioactive isotopes used in radiation therapy include, for example, Ra-226, Co-60, Cs-137, Ir-192 and 1-125. External beam therapy is commonly delivered via a medical linear accelerator or Cobalt-60 unit. An exemplary external beam radiation therapy regimen is 1.8-2 Gy per day, administered 5 days each week for 5-7 weeks, depending on the particular clinical situation, wherein the abbreviation Gy represents a Gray which represents 1 J/kg of tissue. Radiation therapy may be administered in parallel with the other therapeutics, i.e., where relevant, it may be administered as a separate pharmaceutical composition. Radiation therapy may be administered at the same time or sequentially with the other therapeutic agents. The dosage schedule of radiation therapy may be different than the dosage schedule of the other compounds, although overlapping in time. Alternatively, where relevant, the radiotherapeutic may be administered together with the other compounds, i.e., in a single pharmaceutical composition. In some embodiments, the radiation therapy and temozolomide and the VEGFR2 specific inhibitor are administered in parallel within five days of each other, 24 hours, 12 hours, or 6 hours of each other. In an exemplary embodiment, Temozolomide is administered orally, the VEGFR2 specific inhibitor is administered intraperitoneally or intravenously, and radiation therapy is administered via an external beam.

The present invention also includes kits comprising a VEGFR2 specific inhibitor and temozolomide, and instructions for the use thereof. The instructions include instructions for inhibiting the growth of a cancer cell using the combination of the invention and/or instructions for a method of treating a patient having a cancer using a combination of temozolomide and a VEGFR2 specific inhibitor, optionally in combination with radiation therapy.

The elements of the kits of the present invention are in a suitable form for a kit, such as a solution or lyophilized powder. The concentration or amount of the elements of the kits will be understood by the skilled artisan to varying depending on the identity and intended use of each element of the kit.

When a kit is supplied, the different components of the combination may be packaged in separate containers and admixed immediately before use. Such packaging of the components separately may permit long-term storage without losing the active components' functions. The inhibitors may be present a single container.

The reagents included in the kits can be supplied in containers of any sort such that the life of the different components are preserved and are not adsorbed or altered by the materials of the container. For example, sealed glass ampules may contain lyophilized therapeutic agents, or buffers that have been packaged under a neutral, non-reacting gas, such as nitrogen Ampules may consist of any suitable material, such as glass, organic polymers, such as polycarbonate, polystyrene, etc., ceramic, metal or any other material typically employed to hold similar reagents. Other examples of suitable containers include simple bottles that may be fabricated from similar substances as ampules, and envelopes, that may comprise foil-lined interiors, such as aluminum or an alloy. Other containers include test tubes, vials, flasks, bottles, IV bags, syringes, or the like. Containers may have a sterile access port, such as a bottle having a stopper that can be pierced by a hypodermic injection needle. Other containers may have two compartments that are separated by a readily removable membrane that upon removal permits the components to be mixed. Removable membranes may be glass, plastic, rubber, etc.

Kits may also be supplied with instructional materials. Instructions may be printed on paper or other substrate, and/or may be supplied as an electronic-readable medium, such as a floppy disc, CD-ROM, DVD-ROM, Zip disc, videotape, audiotape, flash memory device etc. Detailed instructions may not be physically associated with the kit; instead, a user may be directed to an internet web site specified by the manufacturer or distributor of the kit, or supplied as electronic mail.

The cancers and cells therefrom referred to in the instructions of the kits include glioblastoma, breast cancer, colon cancer, ovarian carcinoma, osteosarcoma, cervical cancer, prostate cancer, lung cancer, synovial carcinoma, pancreatic cancer, melanoma, multiple myeloma, neuroblastoma, and rhabdomyosarcoma.

### Incorporation by Reference

All documents and references, including patent documents and websites, described herein are individually incorporated by reference to into this document to the same extent as if there were written in this document in full or in part.

### EXAMPLES

The invention is now described by reference to the following examples, which are illustrative only, and are not intended to limit the present invention. While the invention has been described in detail and with reference to specific embodiments thereof, it will be apparent to one of skill in the art that various changes and modifications can be made thereto without departing from the spirit and scope thereof.

### Example 1

### Production of VEGFR2-Specific Inhibitor

Compound 1, a VEGFR2 specific inhibitor represented by SEQ ID NO: 4, was expressed in E. coli BL21(DE3) pLysS (Invitrogen, Carlsbad, Calif.) using standard methods, refolded and purified from E. coli inclusion bodies. E. coli cell pellets were resuspended in 50 mM HEPES 500 mM NaCl, 5 mM EDTA and lysed with a M-110EH microfluidizer (Microfluidics, Newton, Mass.). Inclusion bodies were isolated, washed and solubilized with 6M guanidine-HCl, 50 mM Tris (pH 8), 5 mM EDTA and 2 mM tris (2-carboxyethyl) phosphine hydrochloride (TCEP). Compound 1 was refolded by dialysis against 50 mM NaAcOH (pH 4.5) and 0.1 mM TCEP. Compound 1 was purified using a SP-Sepharose column (Amersham Biosciences) with a linear elution gradient of 0-1 M NaCl and 50 mM NaAcOH (pH 4.5), dialyzed against 50 mM NaAcOH (pH 4.5), 100 mM NaCl and concentrated. Compound 1 was then modified with a single PEG molecule using site specific pegylation at the single cysteine at the C-terminal tail of Compound 1 (position 93 of SEQ ID NO: 4). One mg/mL Compound 1 in 50 mM NaAcOH (pH 5.5), 0.5 M arginine, 0.1 mM TCEP was added to a maleimide-conjugated, branched 40 kDa methoxypolyethylene glycol (PEG) (Nektar Therapeutics, Huntsville, Ala.) in 2.5 times molar excess at 25° C. for 1 hr. The reaction was stopped with 10-fold molar excess of β-mercaptoethanol. Compound 1 was further purified by SP-Sepharose as described above.

### Example 2

### Coadministration of Compound 1 and Temozolomide to an Established In Vivo Glioma Model

U87vIIILuc Glioblastoma Xenograft Model

Tumor Implantation. 9 week-old male NOD-SCID mice (Charles River Laboratories, Wilmington, Mass., USA) were anesthetized by 2% isoflurane. 5×10⁴ U87vIIIluc tumor cells were implanted intracranially using the following stereotactic coordinates: 0.5 mm posterior, 2.5 mm lateral, and 3.5 mm intraparenchymal Animal care was kept in strict accordance with the institutional animal care and use committee of Massachusetts General Hospital.

Drug Administration. After randomization, 24 mice were divided into 4 groups (PBS, Compound 1 only, TMZ only, and Compound 1+TMZ) with 6 mice per group. PBS and Compound 1 (60 mg/kg) were delivered intraperitoneally in 200 ul IP volumes three times per week throughout the study until mice reached established criteria for euthanasia. Temozolomide (TMZ) was delivered orally (34 mg/kg) once daily for five days.

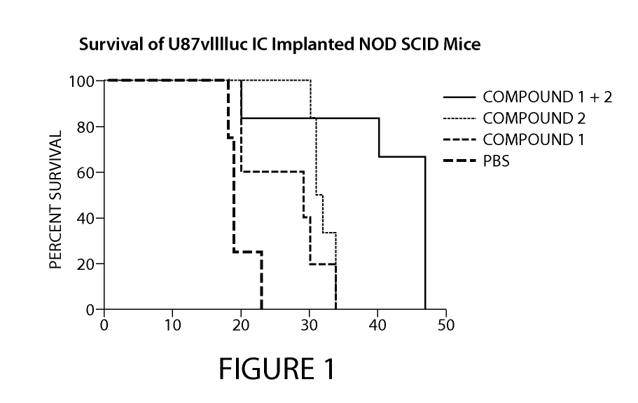

Results are shown in FIG. 1 (Compound 1=VEGFR2 specific inhibitor represented by SEQ ID NO: 4; Compound 2=TMZ; PBS=phosphate buffered saline (vehicle control)). The Y-axis indicates percent survival within each group over the length of the study Animals were followed for survival and sacrificed at the first sign of morbidity. Mean survival was 19, 29, 32 and 47 days for PBS, Compound 1, TMZ, and Compound 1+TMZ, respectively.

### BLI Imaging

Scanning Mice were anesthetized using 1-2% isoflurane before intraperitoneal injection of D-luciferin salt (150 mg/kg in 200 ul PBS; Caliper Life Sciences, Hopkinton Mass.). Bioluminescent imaging (BLI) was performed in a high sensitivity, cooled CCD camera (IVIS®, Xenogen) at five-minute intervals until peak values were recorded for all mice. BLI imaging was performed on days 6, 13, 20 and 26.

Image Analysis. Living Image® software (Xenogen) was used for image capture and signal quantification. Imaging schedules are described in the treatment groups. Statistical analysis was competed using Prism software (Prism 5.0a, Graphpad Software).

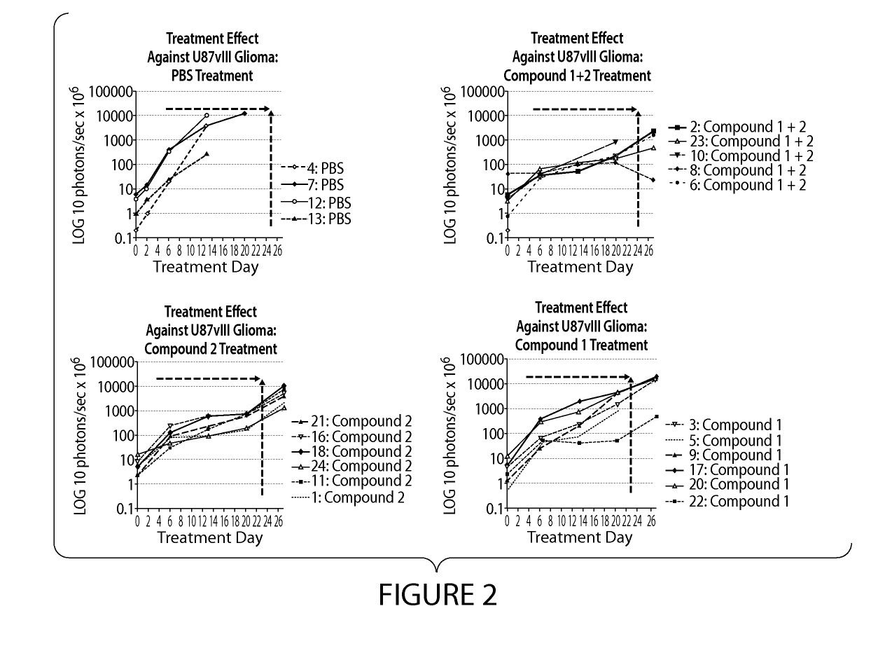

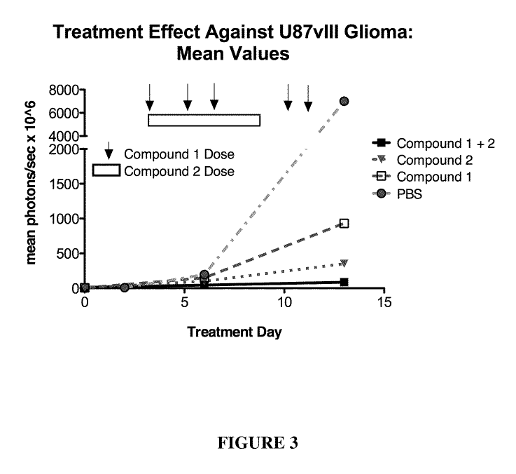

Results are shown in FIGS. 2 and 3 (Compound 1=VEGFR2 specific inhibitor represented by SEQ ID NO: 4; Compound 2=TMZ; PBS=vehicle control). In FIG. 2, convergent arrows indicate maximal BLI as detected for PBS control group. Day 13 mean BLI values (photons/sec×10⁶) increased by 3.64×10⁴ (PBS), 1.79×10² (Compound 1), 6.17×10¹ (TMZ), and 6.46 (Compound 1+TMZ Combo) fold per group. Mean values are shown in FIG. 3.

### Immunohistochemical Staining

Immunohistochemical (IHC) staining was performed on paraffin-embedded mouse brain tissue sections using a polymer peroxidase system (Rat on mouse HRP-Polymer® or Rabbit on Rodent HRP-Polymer®; Biocare Medical, Concord, Calif.). Briefly, tissue sections were deparaffinized, rehydrated and treated with PEROXIDAZED 1® (Biocare Medical) for 5 min to block endogenous peroxidaze activity. To expose antigens, sections were heated under pressure using Decloacking Chamber® (Biocare Medical) with a retrieval solution Reveal® (Biocare Medical) for 40 min and allowed to cool for 10 min. After rinsing in 0.05M tris-buffered saline containing 0.1% tween-20, sections were incubated with a protein blocker (Background Sniper®, Biocare, Medical) for 15 min at room temperature.

Following protein block, sections were incubated with either rabbit polyclonal anti-Ki67 (1:100; Biocare Medical) or rabbbit polyclonal cleaved-caspase 3 (1:100; Biocare Medical) antibodies at room temperature for 30 or 60 min, respectively. Subsequently, sections were incubated with Biocare's Rabbit on Rodent HRP-Polymer (Biocare Medical) for 10 or 30 min, respectively. Thereafter, tissue sections were incubated with DAB chromogen substrate (Biocare Medical) for 5 min in room temperature Finally, sections were lightly counter-stained with Mayer's hematoxylin. For negative control, incubation step with primary antibody was omitted.

IHC staining for CD31 was performed on paraffin-embedded mouse brain tissue sections using Rat on mouse HRP-Polymer® (Biocare Medical). Briefly, sections were deparaffinized, rehydrated and treated with peroxidase (PEROXIDAZED 1, Biocare Medical) for 5 min at room temperature. To expose antigens, sections were treated with trypsin (CAREZYME 1®, Biocare Medical) for 10 min at 37° C. Unspecific protein was blocked by using Rodent Block M® (Biocare Medical) for 30 min. Thereafter, sections were incubated with rat monoclonal anti-CD31 (1:50, Biocare Medical) antibody for 2 h at room temperature. Subsequently, sections were incubated with Rat Probe® (Biocare Medical) for 15 min at room temperature, followed by incubation with Rat-Polymer HRP® (Biocare Medical) for 20 min at room temperature.

To visualize antigens in the tissue, DAB chromogen substrate (Biocare Medical) were used for 5 min Finally, sections were lightly counter-stained with Mayer's hematoxylin. For a negative control, the incubation step with primary antibody was omitted.

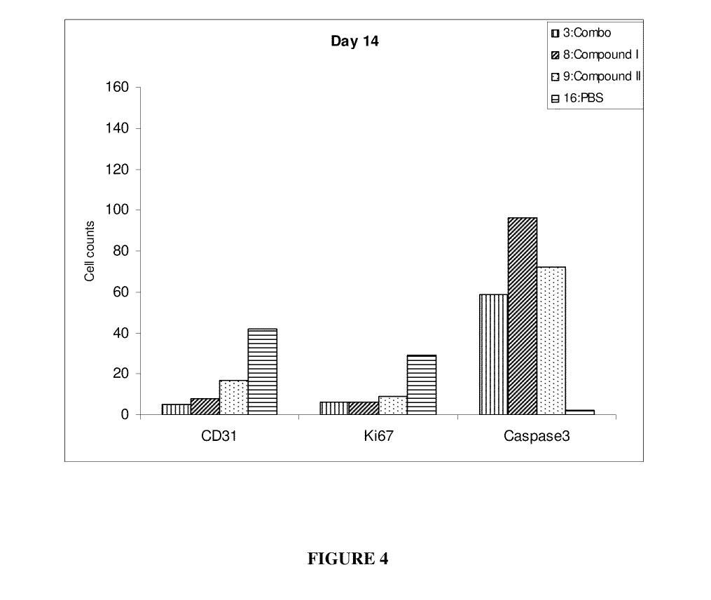

Results are shown in FIG. 4. Immunohistochemical staining against CD31 (endothelial blood vessel formation marker), Ki67 (cell proliferation marker) or cleaved-caspase 3 (cellular apoptosis marker) was performed on paraffin-embedded brain tissue sections. Stained tissue sections were examined with low and high-power light microscopy and the entire tumor area was selected for density counts. Day 14 IHC analysis demonstrated strongly reduced blood vessel formation in Compound 1 and Combo (Compound 1+TMZ) groups compared to TMZ or PBS groups as demonstrated by CD31 staining. Ki67 expression was significantly reduced in all treatment groups compared to PBS control. Enhanced detection of cleaved-caspase 3 was observed in all treatment groups compared to control.

The survival benefit of a combinatorial treatment using Compound 1 and TMZ was superior to that of either agent alone. Tumor burden (BLI, MRI) and IHC analysis suggest that tumor growth suppression plus antiangiogenic effects may underlie the observed enhanced survival of the combo treatment.

### Example 3

### Triple Administration of Compound 1 with Temozolomide and Radiation Therapy to an Established In Vivo Glioma Model

Amplification of the epidermal growth factor receptor (EGFR) and EGFRvIII increase glioma proliferation and invasion properties (Heimberger et al. J Transl Med 2005, 3:38). In addition, pro-survival downstream effectors of this signaling pathway have been demonstrated to be activated in response to radiation in these cells (Lammering et al. J Natl Cancer Inst 2001, 93:921). These characteristics have been associated with the intrinsic resistance of glioblastomas to conventional radiation therapy. However, the 06-methylguanine-DNA methyl-transferase (MGMT) positive expression of U87 cells is also reportedly associated with no response to radiation (Chakravarti et al. Clin Cancer Res 2006, 12:4738). Thus, we wanted to assess whether the predicted survival benefit of a combinatorial treatment with (Compound 1) could be impacted by the activation of radiation-resistance elicited mechanisms.

Tumor implantation was performed as described above in Example 2.

Drug Administration. After randomization, 32 mice were divided into 4 groups (PBS, Radiation Therapy (RT) only, Compound 2 & RT, and Compound 1+Compound 2 & RT) with 8 mice per group. PBS and Compound 1 (60 mg/kg) were delivered intraperitoneally in 200 ul IP volumes three times per week throughout the study until mice reached established criteria for euthanasia. Compound 2 (TMZ) was delivered orally (34 mg/kg) once daily for five days. Compound 2 and RT treatments were initiated after the third dose of Compound 1.

Radiation Therapy. Mice were shielded in a custom-designed block with an aperture of 0.8 cm. After anesthetic induction (ketamine 118 mg/kg i.p.+xylazine 11.8 mg/kg i.p.), mice were irradiated to a total dose of 10 Gy in three daily fractions. 10 Gy were experimentally determined to be the highest tolerable dose for this model.

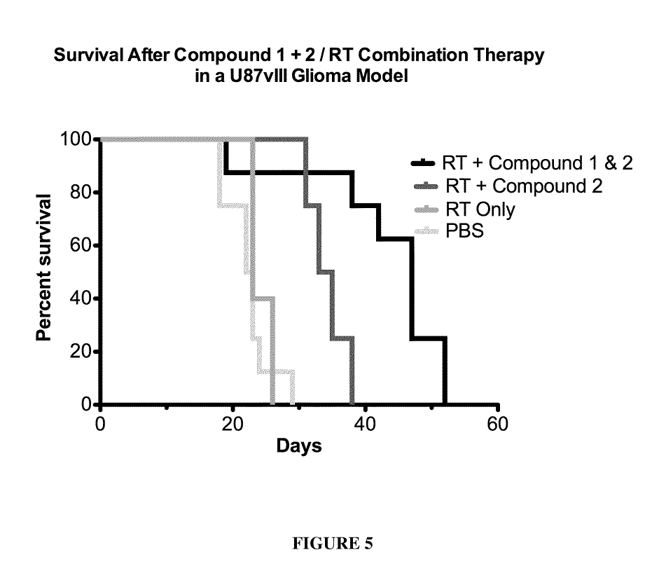

Results are shown in FIG. 5 (Compound 1=VEGFR2 specific inhibitor represented by SEQ ID NO: 4; Compound 2=TMZ; RT=Radiation Therapy; PBS=phosphate buffered saline (vehicle control)). The Y-axis indicates percent survival within each group over the length of the study Animals were followed for survival and sacrificed at the first sign of morbidity. Mean survival was 22, 23, 33 and 47 days for PBS, Radiation Therapy, Compound 2 & RT, and Compound 1+Compound 2 & RT, respectively. Survival of the triple combination treatment was superior to that of the standard of care treatment (TMZ+RT). Survival with the triple combination therapy was significantly greater than survival with PBX, with a p<0.0002 suing a Log-rank Mantel-Cox test.

BLI imaging was performed as described above in Example 2 on days 3, 7, 10, 14, 21, 28, 35, 42, and 49. Image analysis was performed as described above in Example 2.

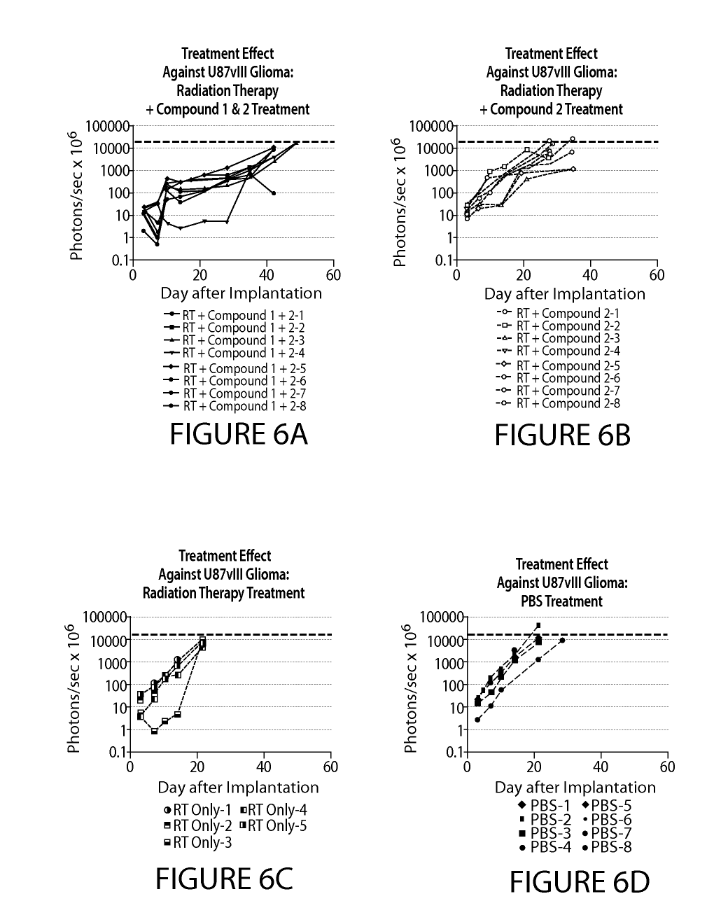

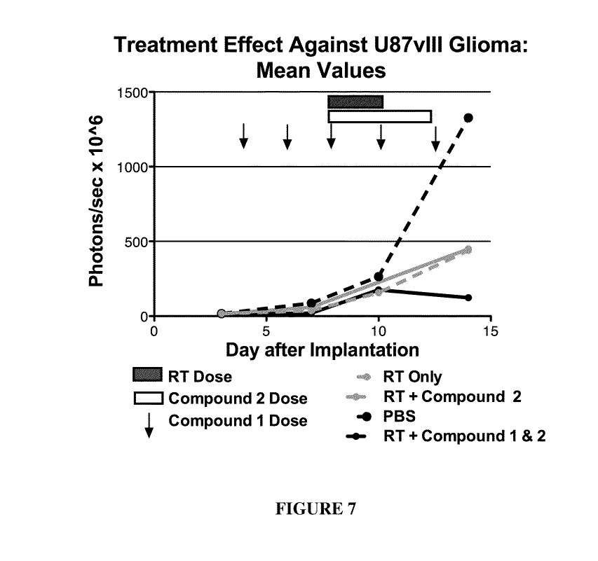

Results are shown in FIGS. 6 and 7 (Compound 1=VEGFR2 specific inhibitor represented by SEQ ID NO: 4; Compound 2=TMZ; RT=Radiation Therapy; PBS=vehicle control). In FIG. 6 shows individual BLI curves and FIG. 7 shows mean values. Day 14 mean BLI values (photons/sec×10⁶) increased by 8.19×10¹ fold (PBS), 2.65×10¹ fold (RT), 2.87×10¹ fold (Compound 2 & RT), and 7.67 fold (Compound 1+Compound 2 & RT). Day 21 mean BLI values (photons/sec×10⁶) increased by 2.83×10² fold (RT), 1.79×10² fold (Compound 2 & RT), and 6.935 fold (Compound 1 plus Compound 2 & RT).

As shown in the figures, the survival benefit of the triple treatment using Compound 1 plus Compound 2 & RT, was superior to that of either RT alone or the standard of care treatment (e.g., RT+TMZ). Mean BLI values, as a measurement of tumor growth, demonstrated a greater tumor control with Compound 1+Compound 2 & RT, when compared to either RT alone or Compound 2 (TMZ) & RT. Mean survival, in days, was comparable between triple and double combination groups from the two individual studies (i.e., double combination Compound 1 plus Compound 2 vs. triple combination Compound 1 plus Compound 2 & RT). These results indicate that Compound 2 did not enhance the response to RT, and that Compound 1 can enhance survival in a radiation insensitive glioblastoma model.

1. A method of treating a subject afflicted with a neoplasm, said method comprising administering to the subject a polypeptide comprising a VEGFR2-binding tenth fibronectin III domain (¹⁰Fn3) together or in parallel with temozolomide in amounts that together are effective to treat said neoplasm.

2. The method of claim 1, wherein the neoplasm is glioblastoma.

3. The method of claim 1 or 2, further comprising administering radiation therapy to the subject.

4. The method of claim 2, wherein the glioblastoma is radiation insensitive.

5. The method of any one of claims 1-4, wherein the polypeptide and temozolomide are administered sequentially.

6. The method of any one of claims 1-5, wherein the VEGFR2-binding ¹⁰Fn3 comprises a BC loop having the amino acid sequence set forth in residues 14-24 SEQ ID NO: 4, a DE loop having the amino acid sequence set for in residues 44-50 of SEQ ID NO: 4, and an FG loop having the amino acid sequence set for in residues 69-82 of SEQ ID NO: 4.

7. The method of any one of claims 1-6, wherein the VEGFR2-binding ¹⁰Fn3 comprises an amino acid sequence at least 90% identical to any one of SEQ ID NOS: 2-62.

8. The method of any one of claims 1-7, wherein the VEGFR2-binding ¹⁰Fn3 is pegylated.

9. A method of reducing the severity, delaying the onset, or preventing the development of VEGFR2 resistance in a subject afflicted with a neoplasm, said method comprising administering a polypeptide comprising a VEGFR2-binding tenth fibronectin III domain (¹⁰Fn3) together or in parallel with temozolomide.

10. The method of claim 9, wherein the neoplasm is glioblastoma.

11. The method of claim 9 or 10, further comprising administering radiation therapy to the subject.

12. The method of claim 10, wherein the glioblastoma is radiation insensitive.

13. The method of any one of claims 9-12, wherein the polypeptide and temozolomide are administered sequentially.

14. The method of any one of claims 9-13, wherein the VEGFR2-binding ¹⁰Fn3 comprises a BC loop having the amino acid sequence set forth in residues 16-23 SEQ ID NO: 4, a DE loop having the amino acid sequence set for in residues 45-49 of SEQ ID NO: 4, and an FG loop having the amino acid sequence set for in residues 70-80 of SEQ ID NO: 4.

15. The method of any one of claims 9-14, wherein the VEGFR2-binding ¹⁰Fn3 comprises an amino acid sequence at least 90% identical to any one of SEQ ID NOS: 2-62.

16. The method of any one of claims 9-15, wherein the VEGFR2-binding ¹⁰Fn3 is pegylated.

17. The method of any one of claims 9-16, wherein the development of VEGFR2 resistance is delayed by at least one week.


SEQUENCE LISTING (71 sequences)

SEQ ID NO: 1 - protein, 94 aa, Homo sapiens
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 2 - protein, 94 aa, Artificial Sequence
  Description of Artificial Sequence Synthetic polypeptide
VSDVPRDLEVVAATPTSLLISWRHPHFPTRYYRITYGETGGNSPVQEFTVPLQPPTATISGLKPGVDYTITVYAVTEGPNERSLFIPISINYRT

SEQ ID NO: 3 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITVYAVTEGPNERSLFIPISINYRT

SEQ ID NO: 4 - protein, 94 aa, Artificial Sequence
  Description of Artificial Sequence Synthetic polypeptide
GEVVAATPTSLLISWRHPHFPTRYYRITYGETGGNSPVQEFTVPLQPPTATISGLKPGVDYTITVYAVTDGRNGRLLSIPISINYRTEIDKPCQ

SEQ ID NO: 5 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITVYAVTDGRNGRLLSIPISINYRT

SEQ ID NO: 6 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTMGLYGHELLTPISINYRT

SEQ ID NO: 7 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTDGENGQFLLVPISINYRT

SEQ ID NO: 8 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTMGPNDNELLTPISINYRT

SEQ ID NO: 9 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTAGWDDHELFIPISINYRT

SEQ ID NO: 10 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTSGHNDHMLMIPISINYRT

SEQ ID NO: 11 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTAGYNDQILMTPISINYRT

SEQ ID NO: 12 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTFGLYGKELLIPISINYRT

SEQ ID NO: 13 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTTGPNDRLLFVPISINYRT

SEQ ID NO: 14 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTDVYNDHEIKTPISINYRT

SEQ ID NO: 15 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTDGKDGRVLLTPISINYRT

SEQ ID NO: 16 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTEVHHDREIKTPISINYRT

SEQ ID NO: 17 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTQAPNDRVLYTPISINYRT

SEQ ID NO: 18 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTREENDHELLIPISINYRT

SEQ ID NO: 19 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTVTHNGHPLMTPISINYRT

SEQ ID NO: 20 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTLALKGHELLTPISINYRT

SEQ ID NO: 21 - protein, 94 aa, Artificial Sequence
  Description of Artificial Sequence Synthetic polypeptide
VSDVPRDLEVVAATPTSLLISWRHPHFPTRYYRITYGETGGNSPVQEFTVPLQPPTATISGLKPGVDYTITGYAVTVAQNDHELITPISINYRT

SEQ ID NO: 22 - protein, 94 aa, Artificial Sequence
  Description of Artificial Sequence Synthetic polypeptide
VSDVPRDLEVVAATPTSLLISWRHPHFPTRYYRITYGETGGNSPVQEFTVPLQPPAATISGLKPGVDYTITGYAVTMAQSGHELFTPISINYRT

SEQ ID NO: 23 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTVERNGRVLMTPISINYRT

SEQ ID NO: 24 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTVERNGRHLMTPISINYRT

SEQ ID NO: 25 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTLERNGRELMTPISINYRT

SEQ ID NO: 26 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTEERNGRTLRTPISINYRT

SEQ ID NO: 27 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTVERNDRVLFTPISINYRT

SEQ ID NO: 28 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTVERNGRELMTPISINYRT

SEQ ID NO: 29 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTLERNGRELMVPISINYRT

SEQ ID NO: 30 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTDGRNDRKLMVPISINYRT

SEQ ID NO: 31 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTDGQNGRLLNVPISINYRT

SEQ ID NO: 32 - protein, 87 aa, Artificial Sequence
  Description of Artificial Sequence Synthetic polypeptide
EVVAATPTSLLISWRHHPHFPTRYYRITYGETGGNSPVQEFTVPLQPPTATISGLKPGVDYTITGYAVTVHWNGRELMTPISINYRT

SEQ ID NO: 33 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTEEWNGRVLMTPISINYRT

SEQ ID NO: 34 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTVERNGHTLMTPISINYRT

SEQ ID NO: 35 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTVEENGRQLMTPISINYRT

SEQ ID NO: 36 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTLERNGQVLFTPISINYRT

SEQ ID NO: 37 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTVERNGQVLYTPISINYRT

SEQ ID NO: 38 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTWGYKDHELLIPISINYRT

SEQ ID NO: 39 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTLGRNDRELLTPISINYRT

SEQ ID NO: 40 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTDGPNDRLLNIPISINYRT

SEQ ID NO: 41 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTFARDGHEILTPISINYRT

SEQ ID NO: 42 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTLEQNGRELMTPISINYRT

SEQ ID NO: 43 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTVEENGRVLNTPISINYRT

SEQ ID NO: 44 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTLEPNGRYLMVPISINYRT

SEQ ID NO: 45 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTEGRNGRELFIPISINYRT

SEQ ID NO: 46 - protein, 94 aa, Artificial Sequence
  Description of Artificial Sequence Synthetic polypeptide
VSDVPRDLEVVAATPTSLLISWRHPHFPTRYYRITYGETGGNSPVQEFTVPLQPPAATISGLKPGVDYTITGYAVTWERNGRELFTPISINYRT

SEQ ID NO: 47 - protein, 94 aa, Artificial Sequence
  Description of Artificial Sequence Synthetic polypeptide
VSDVPRDLEVVAATPTSLLISWRHPHFPTRYYRITYGETGGNSPVQEFTVPLQPPAATISGLKPGVDYTITGYAVTKERNGRELFTPISINYRT

SEQ ID NO: 48 - protein, 94 aa, Artificial Sequence
  Description of Artificial Sequence Synthetic polypeptide
VSDVPRDLEVVAATPTSLLISWRHPHFPTHYYRITYGETGGNSPVQEFTVPLQPPAATISGLKPGVDYTITGYAVTTERTGRELFTPISINYRT

SEQ ID NO: 49 - protein, 94 aa, Artificial Sequence
  Description of Artificial Sequence Synthetic polypeptide
VSDVPRDLEVVAATPTSLLISWRHPHFPTHYYRITYGETGGNSPVQEFTVPLQPPAATISGLKPGVDYTITGYAVTKERSGRELFTPISINYRT

SEQ ID NO: 50 - protein, 94 aa, Artificial Sequence
  Description of Artificial Sequence Synthetic polypeptide
VSDVPRDLEVVAATPTSLLISWRHPHFPTHYYRITYGETGGNSPVQEFTVPLQPPAATISGLKPGVDYTITGYAVTLERDGRELFTPISINYRT

SEQ ID NO: 51 - protein, 94 aa, Artificial Sequence
  Description of Artificial Sequence Synthetic polypeptide
VSDVPRDLEVVAATPTSLLISWRHPHFPTRYYRITYGETGGNSPVQEFTVPLQPPLATISGLKPGVDYTITGYAVTKERNGRELFTPISINYRT

SEQ ID NO: 52 - protein, 94 aa, Artificial Sequence
  Description of Artificial Sequence Synthetic polypeptide
VSDVPRDLEVVAATPTSLLISWRHPHFPTRYYRITYGETGGNSPVQEFTVPLQPTTATISGLKPGVDYTITGYAVTWERNGRELFTPISINYRT

SEQ ID NO: 53 - protein, 94 aa, Artificial Sequence
  Description of Artificial Sequence Synthetic polypeptide
VSDVPRDLEVVAATPTSLLISWRHPHFPTRYYRITYGETGGNSPVQEFTVPLQPTVATISGLKPGVDYTITGYAVTLERNDRELFTPISINYRT

SEQ ID NO: 54 - protein, 95 aa, Artificial Sequence
  Description of Artificial Sequence Synthetic polypeptide
MGEVVAATPTSLLISWRHPHFPTRYYRITYGETGGNSPVQEFTVPLQPPTATISGLKPGVDYTITVYAVTDGRNGRLLSIPISINYRTEIDKPSQ

SEQ ID NO: 55 - protein, 95 aa, Artificial Sequence
  Description of Artificial Sequence Synthetic polypeptide
MGEVVAATPTSLLISWRHPHFPTRYYRITYGETGGNSPVQEFTVPLQPPTATISGLKPGVDYTITVYAVTDGRNGRLLSIPISINYRTEIDKPCQ

SEQ ID NO: 56 - protein, 102 aa, Artificial Sequence
  Description of Artificial Sequence Synthetic polypeptide
MVSDVPRDLEVVAATPTSLLISWRHPHFPTRYYRITYGETGGNSPVQEFTVPLQPPTATISGLKPGVDYTITVYAVTDGRNGRLLSIPISINYRTEIDKPSQ

SEQ ID NO: 57 - protein, 88 aa, Artificial Sequence
  Description of Artificial Sequence Synthetic polypeptide
MGEVVAATPTSLLISWRHPHFPTRYYRITYGETGGNSPVQEFTVPLQPPTATISGLKPGVDYTITVYAVTDGWNGRLLSIPISINYRT

SEQ ID NO: 58 - protein, 88 aa, Artificial Sequence
  Description of Artificial Sequence Synthetic polypeptide
MGEVVAATPTSLLISWRHPHFPTRYYRITYGETGGNSPVQEFTVPLQPPTATISGLKPGVDYTITVYAVTEGPNERSLFIPISINYRT

SEQ ID NO: 59 - protein, 95 aa, Artificial Sequence
  Description of Artificial Sequence Synthetic polypeptide
MVSDVPRDLEVVAATPTSLLISWRHPHFPTRYYRITYGETGGNSPVQEFTVPLQPPTATISGLKPGVDYTITVYAVTEGPNERSLFIPISINYRT

SEQ ID NO: 60 - protein, 94 aa, Artificial Sequence
  Description of Artificial Sequence Synthetic polypeptide
VSDVPRDQEVVAATPTSLLISWRHPHFPTRYYRITYGETGGNSPVQEFTVPLQPPAATISGLKPGVDYTITGYAVTMAQSGHELFTPISINYRT

SEQ ID NO: 61 - protein, 86 aa, Artificial Sequence
  Description of Artificial Sequence Synthetic polypeptide
EVVAATPTSLLISWRHPHFPTRYYRITYGETGGNSPVQEFTVPLQPPTATISGLKPGVDYTITGYAVTVHWNGRELMTPISINYRT

SEQ ID NO: 62 - protein, 94 aa, Artificial Sequence
  Description of Artificial Sequence Synthetic polypeptide
VSDVPRDLEVVAATPTSLLISWRHPHFPTRYYRITYGETGGNSPVQEFTVPLQPPLATISGLKPGVDYTITVYAVTKERNGRELFTPISINYRT

SEQ ID NO: 63 - protein, 86 aa, Artificial Sequence
  Description of Artificial Sequence Synthetic polypeptide
EVVAATPTSLLISWRHPHFPTXYYRITYGETGGNSPVQEFTVPLQPXXATISGLKPGVDYTITGYAXTXXXXXXXXXXPISINYRT

SEQ ID NO: 64 - protein, 11 aa, Artificial Sequence
  Description of Artificial Sequence Synthetic peptide
XGXNXXELXTP

SEQ ID NO: 65 - protein, 11 aa, Artificial Sequence
  Description of Artificial Sequence Synthetic peptide
XERNGRXLXTP

SEQ ID NO: 66 - protein, 11 aa, Artificial Sequence
  Description of Artificial Sequence Synthetic peptide
XGXNXRXLXIP

SEQ ID NO: 67 - protein, 11 aa, Artificial Sequence
  Description of Artificial Sequence Synthetic peptide
XGXNXRXLXIP

SEQ ID NO: 68 - protein, 7 aa, Artificial Sequence
  Description of Artificial Sequence Synthetic peptide
EIDKPSQ

SEQ ID NO: 69 - protein, 7 aa, Artificial Sequence
  Description of Artificial Sequence Synthetic peptide
EIDKPCQ

SEQ ID NO: 70 - protein, 7 aa, Artificial Sequence
  Description of Artificial Sequence Synthetic peptide
PSTSTST

SEQ ID NO: 71 - protein, 10 aa, Artificial Sequence
  Description of Artificial Sequence Synthetic peptide
GSGSGSGSGS